(19) United States
(12) Patent Application Publication
(10) Pub. No.: US 2002/0111828 A1
(43) Pub. Date: Aug. 15, 2002

(54) Method for selling and distributing pharmaceuticals

(76) Inventors: Robert Bloder, Lafayette, NJ (US); Howard J. Weisman, Far Hills, NJ (US)

Correspondence Address: RATNER & PRESTIA, PO Box 980, Valley Forge, PA, 19482, US

(21) Appl. No.: 10/037,763

(22) Filed: Oct. 23, 2001

Related U.S. Application Data
(60) Provisional application No. 60/242,996, filed on Oct. 25, 2000.

(51) Int. Cl. A61B 10/00; G06F 17/60

(52) U.S. Cl. 705/2; 700/231; 128/920

(57) ABSTRACT

The present invention provides a method for distributing pharmaceutical products comprising the steps of developing a disease management program and related materials having specific relevance to a counterpart pharmaceutical product to be distributed in conjunction with the counterpart pharmaceutical product, providing a pharmaceutical product supplier to supply the disease management program related materials directly to a patient in conjunction with the counterpart pharmaceutical product when the patient fills a prescription, communicating to a doctor the disease management program and related materials and the relevance of the program and materials to the counterpart pharmaceutical product. The method includes providing a method of communication between the doctor and a patient for whom the doctor has prescribed the disease management program and its counterpart drug. The invention also includes a method for treating a person with a medical condition using a pharmaceutical product comprising the steps recited above.

This invention claims the benefit of earlier filed copending provisional patent application Serial No. 60/242,996 filed on Oct. 25, 2000.

### FIELD OF INVENTION

This invention relates generally to a method of marketing products and more specifically to a method of selling and distributing pharmaceutical drugs.

### BACKGROUND OF THE INVENTION

The pharmaceutical industry generally relies heavily on mass sales force deployment to promote its products to physicians, pharmacists, and health plans. Since 1995, the number of sales representatives in the top 40 pharmaceutical companies has increased about 50% to approximately 70,000 people. In addition to the large, fixed cost of maintaining a permanent sales force, many companies hire contract sales forces to augment their efforts. Although the sales force has proven to be an effective promotion tool, it is also very expensive. A typical physician call, which lasts ordinarily 3 to 5 minutes, can often cost a pharmaceutical company $250 per call.

The typical objective of sales calls and other related promotional activity is to increase new prescriptions. In the case of chronic disease treatments, prescription refills following an initial trial period make up the majority of a product's revenue stream. As a result of poor compliance, however, as well as low persistency rates, only a small fraction of those prescriptions translate into long term sales. Compliance is generally defined as the number of people who properly follow a treatment regime in terms of daily dosage taken during proper intervals. Similarly, persistency is based on the number of people who continue proper compliance over a period of time, especially where chronic treatments are at issue.

This high cost of personal visits impacts the cost of treating medical conditions (especially chronic conditions). Perhaps even more importantly, it ultimately impacts the likelihood of a patient achieving the desired treatment outcome. FIG. 1 illustrates a typical example of a case where a number of patients having a chronic condition begin treatment of a disease treatable by a pharmaceutical product marketed by pharmaceutical manufacturer 100. In FIG. 1, a hypothetical patient population of 1000 people have a particular condition at the beginning of the process in box 110. Of those 1000 patients, however, only about 50% (or 500 patients) are ultimately diagnosed (for a variety of reasons not everyone is diagnosed, including the fact that some will never even seek medical attention) as illustrated schematically in box 120. Of those 500 who are diagnosed, only perhaps 90% (or 450 patients) will be given a prescription by their doctor for treatment by pharmaceutical manufacturer 100's pharmaceutical drug, as shown in box 130. Of those 450, only perhaps 70% (or 315 patients) will actually fill the prescription provided by their doctor (box 140), and of them, only about 70% (or 221 patients) will use the drug for three months. Of those, only about 40% (or 126) will use the drug for over 1 year, and of them, only about 40% (or 50 patients) will achieve their desired outcome. Thus, it is estimated that only perhaps 50 of the original 1000 patients will continue the regime and achieve their desired outcome through the treatment.

As can be inferred from FIG. 1, money flows to the pharmaceutical manufacturer 100 in all steps from 140 to 170 as patients continue to buy drugs. The long term revenue, however, is focused on steps 150 to 170. Thus, to the extent pharmaceutical manufacturers can focus on compliance (box 140) and persistency (boxes 150-170), revenue increases.

Because the number of patients taking a prescribed drug decreases over time, pharmaceutical manufacturers build this anticipated loss of revenue into their cost structures and respond by focusing increased resources on driving more diagnosed patients to start a particular drug, hoping to generate new prescriptions fast enough to offset the loss of revenue that inevitably occurs downstream of the initial prescription. Considering, however, that the bulk of revenue from chronic conditions is derived from refilled prescriptions, these current pharmaceutical marketing techniques have certain limitations.

As another example, large numbers of patients with conditions that have subtle symptoms such as high cholesterol, hypertension, diabetes or prostate cancer do not get diagnosed each year due to inconsistent screening efforts. For example, according to the American Diabetes Association, only 50% of diabetes patients are properly diagnosed in the United States. As discussed above with regard to FIG. 1, for patients who are properly screened, approximately 30% choose not to purchase the prescribed treatment (lack of compliance). Up to one-half of patients discontinue product use within the first six months and only one-third make it through one year (loss of persistency). This lack of compliance and loss of persistency is particularly common among patients who are treated for chronic diseases. The social costs of this phenomenon have been estimated to be over $111 billion each year in increased office visits, hospitalizations, loss of work and invasive procedures as remedies. From a pharmaceutical manufacturer's perspective, the lost revenues from both the unfilled new prescriptions and lapsed refilled prescriptions is likely on the order of several billion dollars.

Because of the relationships shown in FIG. 1, a modest increase of diagnosis, compliance, and/or persistency rates in chronic disease patients can have a very significant impact on drug sales. For example, approximately $5.6 billion of prescription products are used to lower cholesterol in the United States each year. Of the population that has high cholesterol, the current diagnosis rate is estimated to be only 50%. For those who actually begin taking a drug, the persistency rate falls to 40% after the first six months. An increase of just 5% over current persistency rates can create incremental sales of 10% or $574 million in this one class of products alone. In addition to added revenue benefits to the manufacturers, improving persistency lowers alternative expenses to society later in the disease's progression (such as invasive procedures, debilitation that results in a loss of the ability to be productive in society and potentially earn a living, etc.).

Often, the expense of maintaining a standing sales force can only be justified by the continuing growth of successful pharmaceutical drugs. When the sales of a particular product begin to slow, the expense of personal sales people begins to place pressure on net profits, which often requires a reallocation of sales resources in favor of higher growth opportunities. This reallocation causes further declines of those mature products leading to another cycle of sales force cost reductions until the products reach patent expiration. To compensate for this loss of unit sales, most pharmaceutical companies raise the price of their products to recoup this loss. This pattern of resource allocation is repeated for all products in a manufacturer's portfolio of pharmaceutical drugs as they seek to use one of their most expensive resources, the sales force, to promote the products with the highest growth potential.

There is also a trend in the growth of consumer participation in the pharmaceutical consumption market. Consumers are generally assuming an increasingly active role in their own healthcare due to the Internet and the proliferation of other healthcare information services. In the first half of 2000, according to one online market service firm, more than 40 million adults in the United States used the Internet to access health and medical information.

Because consumers have more information available to them now than in the past, consumers are often demanding that their physicians, employers, managed care organizations, and government give them access to better health benefits at lower cost. This new activism among patients, coupled with the increase in the number of consumers as “baby boomers” age, leads to a higher demand for the personalization of healthcare information. But with this demand for personalization of healthcare information, tailored to each individual consumer, comes the need for tools which deliver the information.

### SUMMARY OF INVENTION

The present invention provides a method of increasing compliance and persistency among patients who are prescribed pharmaceutical products. More specifically, the present invention allows for the tracking of, and the gathering of data from, patients who choose to participate in the tacking and data gathering. Specifically, the present invention provides a method for distributing pharmaceutical products (especially prescription drugs) comprising the step of developing a disease management program and/or related materials to be distributed in conjunction with a counterpart pharmaceutical product directly to a patient when the patient fills a prescription. Included in the method is the step of providing a pharmaceutical supplier to supply the disease management program related materials in conjunction with the counterpart pharmaceutical product. The method also includes the step of communicating to a doctor the disease management program (or related materials) and the relationship between the management program and its counterpart drug. Optionally, the method includes the tracking and data compilation of patient progress through doctor/patient/method provider communication, typically via the Internet.

The invention also includes a method of treating a person comprising the steps of developing a disease management program and/or related materials to be distributed in conjunction with a pharmaceutical product when the patient fills a prescription, prescribing the pharmaceutical product and the disease management program's related materials to a patient, providing a pharmaceutical supplier to supply the disease management program in conjunction with the counterpart pharmaceutical product, and then communicating with the patient after the patient begins treatment with the pharmaceutical product and the disease management program. Typically, the means of communication is a global computer network, such as the world wide web or the Internet. Optionally included in this method is the compilation of data gathered through these communications with the patient.

### BRIEF DESCRIPTION OF THE FIGURES

FIG. 1 represents a schematic example of what typically happens when a number of patients having a chronic condition begin treatment by a pharmaceutical product; and

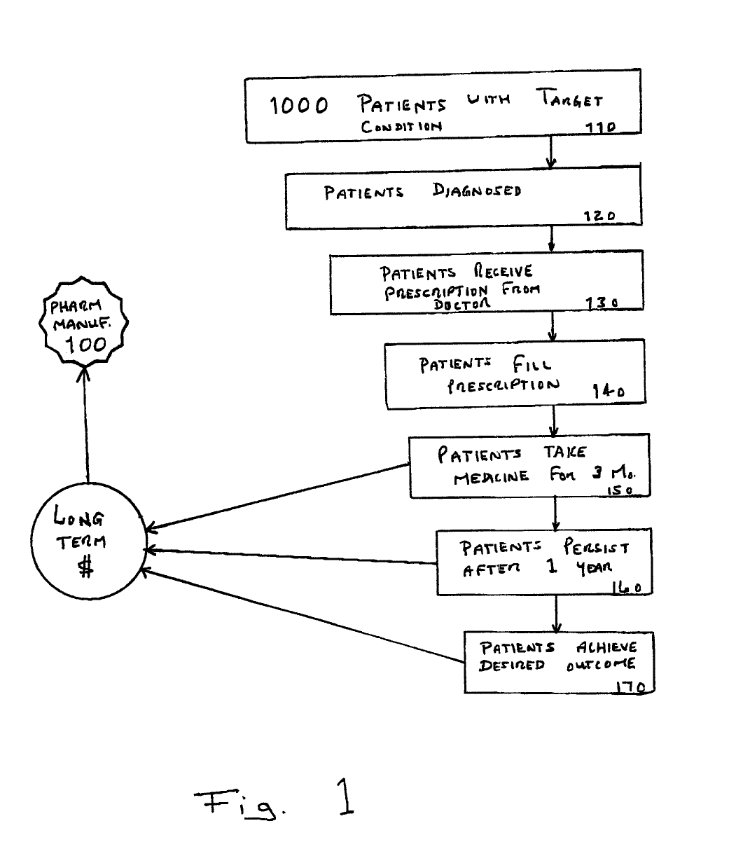

FIG. 2 is a flow chart representing an exemplary process utilizing the method of the present invention.

### DETAILED DESCRIPTION OF THE INVENTION

Traditional pharmaceutical marketing has focused on generating new prescriptions and as such, the majority of marketing resources are allocated to that task. Although it is generally recognized that prescriptions are lost due to loss of compliance and lack of persistency, not much attention has been paid to capture those lost sales. The present invention focuses on the idea that marketing resources must directly target individual patients, because once the prescription is written, the patient is the one who is responsible for actively seeking and maintaining his or her treatment.

Specifically, the present invention provides a method for distributing pharmaceutical products (especially prescription drugs) in conjunction with a disease management program. The disease management program can take many forms (discussed in more detail below), but the key is that the pharmaceutical product is linked to the program such that benefits are derived both by the patient and the pharmaceutical suppliers through data collection and compilation. The method begins with the development of a disease management program and/or related materials to be distributed in conjunction with a counterpart pharmaceutical product.

The disease management program is specific for each pharmaceutical product. The program materials may include both physical analytical equipment, where appropriate, as well as educational materials and analytical forms and voluntary reporting forms and questions. For example, where diabetes is the disease involved, self-diagnostic kits for at-home blood glucose analysis may be a part of the materials for the management program. Conversely, where diabetes is not the disease of interest, but hypertension is, blood pressure measurement would be a part of the related materials distributed under the hypertension management program. More generally, the materials to be distributed along with the drug would include, where appropriate, such things as surrogate marker tests for acute or chronic conditions, patient questionnaires, surveys, quality of life questions, health risk assessment tests, and reporting directions or instructions.

These materials would be designed both to educate and encourage continued use of the drug. This is especially important for disease states such as diabetes or high cholesterol, where long term treatment is often necessary. As discussed above, it may be that a patient does not “feel” bad when his cholesterol is too high or his blood pressure is too high, and thus is not encouraged to comply with the ongoing use of the prescribed pharmaceutical. Through the disease management program and/or related materials, several types of encouragement can be provided. These encouragement would include such simple things as educational reminders, charts to fill in on a daily basis when the patient takes his blood pressure, or simply monthly regimes of reporting to his doctor, say via the Internet, his average insulin level over the course of that month. These are only examples of the types of interaction that can occur to encourage continued persistency with a particular drug.

Included in the method is the step of providing a pharmaceutical supplier to supply the disease management program's related materials in conjunction with the counterpart pharmaceutical product. Typically, the producer of the disease management program and its related materials makes arrangements with particular pharmacies, pharmaceutical supply houses, or other global communication companies to include the disease management materials with the particular prescribed drug so that when a patient seeks to fill his prescription he can receive the drug and the management program materials simultaneously. It is feasible that all or nearly all pharmacies in the country be provided with the necessary materials, but it is also possible that only a few of the largest prescription fillers be provided with the materials. A typical supplier would be a large, mail or Internet order supplier. In this way, when a patient receives the prescription for the particular drug and its related disease management program, the patient can receive, through a normal course of prescription filling behavior, both the drug and the necessary materials.

Other types of pharmaceutical product suppliers could be utilized in accordance with the present invention. These would include hospitals, doctors, clinics, nursing homes, government agencies, and pharmaceutical manufacturers.

The method also includes the step of communicating to a doctor the disease management program and the program's relationship with its counterpart drug. This step is important because the doctor needs to be aware of the program and its benefits before he can prescribe it or make it available to his patients. Through education by the provider of the method of this invention, the doctor can understand the benefits of applying it to certain patients, particularly those patients who might be more at risk to fall victim to poor compliance or persistency. Once the doctor understands the method and the benefits of the disease management program, he is better able to ask his patients whether they want to partake in the program.

The tracking and data compilation step of the method occurs by providing a method of communication between the doctor and a patient for whom the doctor has prescribed and its counterpart drug. One embodiment of the present invention would utilize a global computer network, such as the world wide web or, more generally, the Internet. The provider of this method may have an electronic presence on the Internet in the form of a computer readable page whereby the patient may report, either anonymously or otherwise, his or her progress or data, such as blood pressure readings over the course of the first three months of use of a particular anti-hypertensive drug. The accumulation of this data would help in a demographic analysis of patients, including the effectiveness not only of the particular drug but also of the method of this invention as it relates to increased compliance and persistency.

There are several such means for providing communication between patients, doctors, and the pharmaceutical product supplier. Typically, a question box and writing area are displayed on the user's screen. The communication provider, such as the web site host, presents boxes or blanks where the user can input information, such as name, address, gender, doctor, etc., and any other relevant information. Alternatively, the process of entering information can utilize clicking on particular icons such as one reading “submit report to doctor” displayed next to the user's writing area. Moreover, the means for fostering information transfer can utilize any combination of text entry (i.e. through a keyboard) and mouse clicks on particular icons. The method provider then receives the user's information at the method provider's server via an electronic communication from the user's computer. This begins the compilation of data and transfer of the data to the ultimate recipient. The ultimate recipient is the doctor in the case where a patient enters reporting information. Similarly, the ultimate recipient is the patient in the case where a doctor responds to the patient's report.

Key in the compilation step is the gathering of information as it flows through the method provider's server. Appropriate safeguards are implemented to protect identities and privileges, and the gathering of this data is done with the knowledge of the parties involved.

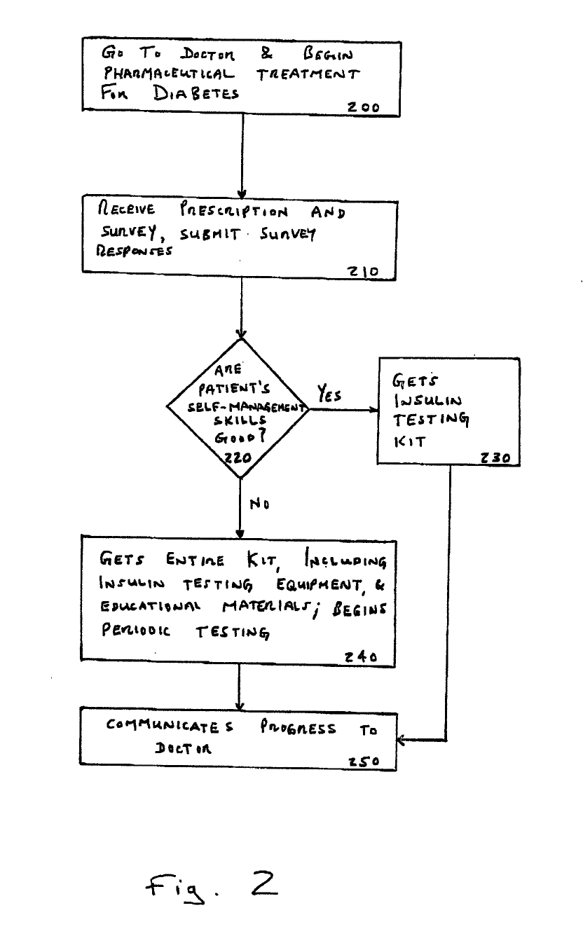

FIG. 2 illustrates an example of the steps involved with one embodiment of the present invention. FIG. 2 uses, as an example, a drug for the treatment of diabetes. In this case, a patient who has diabetes and visits his doctor in step 200. The doctor prescribes a diabetes treatment drug and its counterpart program. The patient then receives the prescription and receives a survey (which may be simultaneous) as shown schematically in step 210. In this example, the survey is used to determine the likelihood that the particular patient would demonstrate high persistency with the drug. The survey would have been created ahead of time and might ask questions such as “have you ever been on a long term pharmaceutical before?” or “have you used it persistently for the last 5 years?” This type of question, and other such questions developed by an appropriate professional, could lead to a determination of whether the particular patient has adequate self-management skills. In an alternative embodiment, the preliminary survey might be forgone and the entire kit might be provided at the time the drug prescription was filled.

In the example of FIG. 2, after the patient submits the completed survey, the results are analyzed by a qualified professional and the results communicated to a patient who may choose to send it to other parties including, medical parties responsible for his/her care. The patient may submit his completed survey, depending upon the particular embodiment, through such channels as regular mail, voice phone, facsimile, or the Internet. If it were through the Internet, the provider of the method of the present invention would have some type of computer readable page on a site accessible by the patient. Through this secure interaction, the patient can submit his completed survey. After the results are analyzed as demonstrated in step 220, the provider of the method might make a determination that a particular patient meets at least a minimum level of self-management skills and therefore does not need a particularly large amount of help. In such a case, the patient might then receive only an blood glucose test kit, as shown in box 230.

On the other hand, if the method provider determines, based on some pre-established criteria, that the patient needs additional help, the entire kit may be provided. In an alternative embodiment, there may be several different versions of a particular kit, depending on the type of patient. Thus, each drug may have several different disease management kits and/or other related information available in many presentations. The doctor or the kit provider, or both of them in combination, may determine the particular kit each patient gets. Moreover, the method of the invention includes the development of a plurality of different management programs for each counterpart drug whereby the doctor can select which of the plurality of management programs should be prescribed along with a particular drug.

As shown in step 240, the patient can begin the particular drug therapy, read the related educational materials, and, in this case, fill out daily blood glucose level data on a form provided in the kit. This data might then be submitted to his doctor on a weekly or monthly basis via a variety of channels, shown as step 250. These channels would include regular mail, telephone, facsimile, or the Internet. In the case of Internet submission, it is possible that the kit provider would have a computer readable and writeable form on its web site such that the patient can fill out an electronic form and submit the data electronically. Once the doctor reviews the information, further follow-ups, either in person or through any of the above mentioned channels of communication, can be suggested by the doctor.

The communication, particularly if it is through the Internet, may be coordinated by the method provider, acting as a host. The patient may electronically transfer his information to the method provider's computer server, which then transmits the information to the particular doctor. In the same fashion, the doctor may communicate back to the patient a message related to the drug performance, via the provider's server. Alternatively, the doctor and patient may interact directly, through traditional electronic mail means. In the former case, the provider's server may act to filter some of the data before transmitting any of it to a third party, an aspect of the invention which is discussed further below. In this situation, identities and any other sensitive or otherwise confidential information can be eliminated before sending performance data and anonymous information to the third parties.

Sometimes, the level of the patient's self-management skills are irrelevant because part of the use of the present method, in addition to increasing persistency, is to compile data and track the effectiveness and effective use, demographically, of particular treatments. Thus, continued reporting and data gathering through patient communications of behavior and self-analysis reporting can be important no matter the level of self-management the patient might demonstrate.

Once the patient and doctor have established a routine regime, the patient's progress can be tracked and adjusted accordingly, all without necessarily having office visits. Another aspect of the present invention, however, occurs separately and involves the gathering or compiling of patient data through the patient's reporting of his or her data to the doctor. This element of communication exists between the doctors and third parties such as the pharmaceutical companies, marketing companies, and the like.

The data of patient practices, persistency, and results can be submitted from the patient to the doctor and then through to the third parties, or it can be submitted directly from the patient to the third parties simultaneous to the patient-doctor communications. The data obtained through the tracking of patient persistency and results may be made available, through the provider of the method of the present invention, to such third parties as related healthcare professionals such as clinicians, nurses, other doctors, etc., pharmaceutical manufacturers, marketing consultants or companies, government agencies, and any other party who might gain benefit from the data.

Although the present invention has been particularly described in conjunction with specific preferred embodiments, it is evident that many alternatives, modifications, and variations will be apparent to those skilled in the art. It is therefore contemplated that the appended claims will embrace any such alternatives, modifications, and variations as falling within the true scope and spirit of the present invention.

What is claimed:

1. A method for distributing pharmaceutical products comprising the steps of:
developing a disease management program and related materials having specific relevance to a counterpart pharmaceutical product to be distributed in conjunction with said counterpart pharmaceutical product;
providing a pharmaceutical product supplier to supply said disease management program related materials directly to a patient in conjunction with said counterpart pharmaceutical product when said patient fills a prescription;
communicating to a doctor said disease management program and said related materials and said relevance of said program and said materials to said counterpart pharmaceutical product; and
providing means of communication between said doctor, a patient for whom said doctor has prescribed said disease management program and its counterpart drug, and said pharmaceutical product supplier.

2. The method of claim 1 wherein said step of developing said disease management program includes developing a plurality of different management programs for each counterpart drug whereby said doctor can select which of said plurality of management programs should be prescribed along with said drug.

3. The method of claim 1 wherein said disease management program includes a diagnostic test for an acute or chronic disease.

4. The method of claim 1 further comprising the step of preventing a patient's participation in said disease management program until said patient has received said disease management program materials and said counterpart pharmaceutical product.

5. The method of claim 1 further comprising the step of providing an electronic host with a computer readable site on a global computer network for said patient to communicate with said host.

6. The method of claim 1 further comprising the step of compiling data from said patient.

7. The method of claim 6 wherein said compiling step is performed via communication with said patient via a global computer network.

8. The method of claim 1 further wherein said means of communication comprises a computer accessible site on a global computer network.

9. The method of claim 1 wherein said means of communication includes the use of a global computer network.

10. The method of claim 1 further comprising the step of providing prescription adherence reminders after said patient fills said prescription.

11. The method of claim 1 further comprising the step of waiting until said patient fills said prescription and begins following said disease management program and then contacting said patient for feedback.

12. The method of claim 11 further comprising the step of sharing said feedback with a pharmaceutical product manufacturer.

13. The method of claim 11 further comprising the step of using said feedback to market pharmaceutical products.

14. A method for distributing pharmaceutical products comprising the steps of:
developing a disease management program and related materials to be distributed in conjunction with a counterpart pharmaceutical product;
providing a pharmaceutical supplier to supply said disease management program related materials in conjunction with said counterpart pharmaceutical product;
communicating to a doctor said disease state management program and said program's relationship with its counterpart drug;
not allowing participation of said patient in said disease management program until after said patient has filled said prescription for said disease management program and its counterpart drug; and
providing a method of communication between said doctor and a patient for whom said doctor has prescribed said disease management program and its counterpart drug.

15. A method of treating a person with a medical condition using a pharmaceutical product comprising the steps of:
developing a disease management program and related materials to be distributed in conjunction with a pharmaceutical product;
prescribing said pharmaceutical product and said disease management program related materials to a patient;
providing a pharmaceutical supplier to supply said disease management program in conjunction with said counterpart pharmaceutical product directly to a patient in conjunction with said counterpart pharmaceutical product when said patient fills a prescription;
communicating with said patient after said patient begins treatment with said pharmaceutical product and said disease management program via a global computer network; and
compiling data from said communications with said patient.

16. The method of claim 15 wherein said patient chooses to participate in said disease management program in addition to receiving said pharmaceutical product before said prescription is written.
